(19) United States
(12) Patent Application Publication
(10) Pub. No.: US 2020/0132670 A1
(43) Pub. Date: Apr. 30, 2020

(54) FATTY LIVER AND LIVER FIBROSIS EVALUATION SYSTEM BASED ON IMPEDANCE

(71) Applicant: KOREA RESEARCH INSTITUTE OF CHEMICAL TECHNOLOGY, Daejeon (KR)

(72) Inventors: Jung-Hwa Oh, Daejeon (KR); Jaehwan Ahn, Daejoen (KR); Seokjoo Yoon, Daejoen (KR); Jun-Ho Ahn, Daejeon (KR); Heeyoung Yang, Daejeon (KR); Hyoung-Yun Han, Daejeon (KR)

(73) Assignee: KOREA RESEARCH INSTITUTE OF CHEMICAL TECHNOLOGY, Daejeon (KR)

(21) Appl. No.: 16/393,684

(22) Filed: Apr. 24, 2019

(30) Foreign Application Priority Data
Oct. 18, 2018 (KR) 10-2018-0124677
Mar. 19, 2019 (KR) 10-2019-0031120

(51) Int. Cl. G01N 33/50; G01N 27/02

(52) U.S. Cl. CPC G01N 33/5014; G01N 27/026; G01N 33/5067

(57) ABSTRACT

The present invention relates to a fatty liver and liver fibrosis evaluation system based on impedance. The fatty liver and liver fibrosis evaluation system based on impedance provided in one aspect of the present invention uses 3-dimensional liver microtissues so that the fatty liver formation and the stiffness changes resulted from liver fibrosis induced by a test drug can be analyzed with non-invasive and highly reproducible manner.

### CROSS REFERENCE TO RELATED APPLICATION

This patent application claims the benefit of priority under 35 U.S.C. § 119 from Korean Patent Application No. 10-2018-0124677, filed Oct. 18, 2018 and Korean Patent Application No. 10-2019-0031120, filed Mar. 19, 2019, the contents of which are incorporated herein by reference.

### BACKGROUND OF THE INVENTION

### 1. Field of the Invention

The present invention relates to a fatty liver and liver fibrosis evaluation system based on impedance.

### 2. Description of the Related Art

The liver goes through steps such as fatty liver, liver fibrosis, and cirrhosis until liver cancer develops (non-patent reference 1, Liver Cancer. 2013 August; 2(3-4): 365-366). Therefore, it is very important to screen a drug causing liver fibrosis in the early stage of new drug development.

In the early stage of screening, human-derived cells are cultured in a culture dish and then exposed directly to a drug, followed by investigation of cellular activity to judge whether the activity decreases or not. However, the result of this investigation is not reliable, compared with the clinical test result such as drug metabolism. To overcome the problem above, cells were cultured three-dimensionally to form small tissues such as micro-tissue (MT) and/or cell spheroid, followed by screening. In that case, the drug metabolizing enzyme or the activity is higher than the liver cells cultured in a culture dish. Therefore, the method above is widely used as an alternative test method. However, the process of cell lysis or section is necessary, inconveniently, to screen not only a drug causing toxicity but also for those drugs inducing intracellular fat accumulation or liver fibrosis by inducing over-secretion of collagen, a long fibrous protein. To avoid such an inconvenient process in the investigation of the internal state of the tissue, impedance spectroscopy letting alternating currents with various frequencies flow has been studied.

Cell membrane is composed of a lipid layer which is either hydrophobic or hydrophilic, by which the cell membrane has the characteristics of a capacitor. The capacitor acts as a low frequency filter that passes high frequency currents but blocks low frequency currents.

This allows us to evaluate the internal state of the tissue through the difference between high and low frequency impedances (values that interfere with current flow when a voltage is applied across the circuit). The internal state of the tissue can be evaluated through the difference of impedance (the value that interrupts current flow when a voltage is applied in the circuit) between high frequency and low frequency. More than 70% of a healthy tissue is composed of ionic water which allows electric current well. However, in the tissue where fat is formed by a drug, the current resistance increases so that the internal impedance increases.

When liver fibrosis occurs in the liver tissue, fibrous protein increases between cells, resulting in the increase of tissue stiffness. When the tissue was placed in a narrow passage with a constant pressure, the resistance of the tissue increased, which was correlated with the tissue strain. The stiffness of the tissue can be measured through the ratio of the applied pressure to the strain.

Based on the previous studies in relation to the above, the present inventors developed a fatty liver and liver fibrosis evaluation system based on impedance that can analyze fatty liver and/or liver fibrosis caused by a drug under screening with non-invasive and highly reproducible manner using micro-tissue and/or cell spheroid, and accordingly presented a method for evaluating fatty liver and liver fibrosis using the system above.

### PRIOR ART REFERENCE

### Non-Patent Reference

(Non-Patent Reference 1) Liver Cancer. 2013 August; 2(3-4): 365-366

### SUMMARY OF THE INVENTION

It is an object of the present invention, according to an aspect of the present invention, to provide a fatty liver and liver fibrosis evaluation system based on impedance that can analyze drug-induced fatty liver and liver fibrosis by using 3-dimensional liver microtissues with non-invasive and highly reproducible manner.

It is another object of the present invention, according to another aspect of the present invention, to provide a method for evaluating the test drug induced fatty liver by using the fatty liver and liver fibrosis evaluation system based on impedance above.

It is also an object of the present invention, according to another aspect of the present invention, to provide a method for evaluating the test drug induced liver fibrosis by using the fatty liver and liver fibrosis evaluation system based on impedance above.

To achieve the above objects, the present invention, according to an aspect of the present invention, provides a fatty liver and liver fibrosis evaluation system based on impedance which comprises a liver cell spheroid strain generating part comprising a first tube; and a second tube inserted into the first tube and having a smaller length than that of the first tube; and an impedance measuring part comprising an impedance analyzer connected to the liver cell spheroid strain generating part to measure impedance of liver cell spheroid; and evaluating fatty liver and liver fibrosis through the impedance measured in the impedance measuring part.

In another aspect of the present invention, the present invention provides a method for evaluating the test drug induced fatty liver comprising the following steps:

placing the liver cell spheroid treated with a test drug in the inside of the first tube of the fatty liver and liver fibrosis evaluation system based on impedance above;

locating the liver cell spheroid to contact with an end of the second tube and measuring the first impedance by using the impedance analyzer;

removing the liver cell spheroid from the second tube and measuring the second impedance by using the impedance analyzer;

obtaining the interior resistance, the exterior resistance and the capacitance of the liver cell spheroid based on the first impedance and the second impedance measured above; and

evaluating the test drug induced fatty liver by comparing the results above with those of the normal control.

In addition, the present invention provides, in another aspect of the present invention, a method for evaluating the test drug induced liver fibrosis comprising the following steps:

placing the liver cell spheroid treated with a test drug in the inside of the first tube of the fatty liver and liver fibrosis evaluation system based on impedance above;

locating the liver cell spheroid to contact with an end of the second tube and measuring the first impedance by using the impedance analyzer;

pushing the liver cell spheroid in the inside of the second tube and measuring the second impedance by using the impedance analyzer;

pushing the liver cell spheroid in the inside of the second tube and measuring the third impedance by using the impedance analyzer;

removing the liver cell spheroid from the second tube and measuring the fourth impedance by using the impedance analyzer;

obtaining the interior resistance, the exterior resistance and the capacitance of the liver cell spheroid based on the first impedance, the second impedance, the third impedance and the fourth impedance measured above; and

obtaining the stiffness of the spheroid from the pressure changes.

Particularly, the present invention provides, in an aspect of the present invention, a fatty liver and liver fibrosis evaluation system based on impedance which comprises a cylindrical first tube; a cylindrical second tube inserted in the first tube and having an outer surface in contact with the inner surface of the first tube and having a smaller length than that of the first tube; a Y-shaped third tube which is connected to one end of the first tube and comprises a i tube connected to one end of the first tube, a ii tube connected to a syringe to adjust the position of the liver cell spheroid in the first tube by applying pressure in the first tube, and a iii tube having a coil-shaped gold (Au) wire installed in the inside and one end of the gold wire is protruded to the outside; a Y-shaped fourth tube which is connected to another end of the first tube and comprises a I tube connected to another end of the first tube, a II tube connected to a barometer and a syringe pump through the tube, and a III tube having a coil-shaped platinum (Pt) wire installed in the inside and one end of the platinum wire is protruded to the outside; and an impedance analyzer including a working electrode connected to the gold wire protruded outwardly and a counter electrode connected to the platinum wire protruded outwardly.

In another aspect of the present invention, the present invention provides a method for evaluating the test drug induced fatty liver comprising the following steps:

placing the liver cell spheroid treated with a test drug in the inside of the first tube of the fatty liver and liver fibrosis evaluation system based on impedance above;

placing the liver cell spheroid in contact with one end of the second tube using the syringe connected to the ii tube;

locating the liver cell spheroid in the inside of the second tube using the syringe pump connected to the II tube and measuring the first impedance by using the impedance analyzer;

removing (detaching) the liver cell spheroid from the second tube using the syringe pump connected to the II tube and measuring the second impedance by using the impedance analyzer;

obtaining the interior resistance, the exterior resistance and the capacitance of the liver cell spheroid based on the first impedance and the second impedance measured above; and

evaluating the test drug induced fatty liver by comparing the results above with those of the normal control.

In addition, the present invention provides, in another aspect of the present invention, a method for evaluating the test drug induced liver fibrosis comprising the following steps:

placing the liver cell spheroid treated with a test drug in the inside of the first tube of the fatty liver and liver fibrosis evaluation system based on impedance above;

placing the liver cell spheroid in contact with one end of the second tube using the syringe connected to the ii tube;

fixing the liver cell spheroid in one end of the second tube using the syringe pump connected to the II tube and measuring the first impedance by using the impedance analyzer;

moving the liver cell spheroid into the inside of the second tube using the syringe pump connected to the II tube by increasing the pressure and measuring the second impedance by using the impedance analyzer;

further moving the liver cell spheroid into the inside of the second tube using the syringe pump connected to the II tube by increasing the pressure and measuring the third impedance by using the impedance analyzer;

removing the liver cell spheroid from the second tube using the syringe pump connected to the II tube and measuring the fourth impedance by using the impedance analyzer;

obtaining the interior resistance, the exterior resistance and the capacitance of the liver cell spheroid based on the first impedance, the second impedance, the third impedance and the fourth impedance measured above; and

obtaining the stiffness of the spheroid from the pressure changes.

### Advantageous Effect

The fatty liver and liver fibrosis evaluation system based on impedance provided in one aspect of the present invention uses 3-dimensional liver microtissues so that the test drug induced fatty liver and liver fibrosis can be analyzed with non-invasive and highly reproducible manner.

### BRIEF DESCRIPTION OF THE DRAWINGS

FIG. 1 is a schematic diagram illustrating the image of the fatty liver and liver fibrosis evaluation system based on impedance provided in one aspect of the present invention.

FIGS. 2a and 2b are a set of photographs and graphs showing the changes in deformation, impedance, and phase angle of the liver microtissue (MT) according to the pressure changes wherein FIG. 2a illustrates the control group and FIG. 2b illustrates the liver microtissue treated with 0.5 M oleic acid, the fatty liver inducing drug.

FIG. 3 is a graph illustrating the resistance pattern of the liver microtissue (MT) according to the pressure changes.

### DESCRIPTION OF THE PREFERRED EMBODIMENTS

Hereinafter, the present invention is described in detail.

In one aspect of the present invention, the present invention provides a fatty liver and liver fibrosis evaluation system based on impedance which comprises a liver cell spheroid strain generating part comprising a first tube; and a second tube inserted into the first tube and having a smaller length than that of the first tube; and an impedance measuring part comprising an impedance analyzer connected to the liver cell spheroid strain generating part to measure impedance of liver cell spheroid; and evaluating fatty liver and liver fibrosis through the impedance measured in the impedance measuring part.

The evaluation system of the present invention facilitates the observation of the internal state of the microtissue by the steps of fixing the liver cell spheroid (or microtissue, MT) treated with a test drug by applying pressure and flowing a weak alternating current therein. Impedance of the microtissue having a plenty of fat with a high current resistance caused by a fatty liver inducing drug is higher than that of the normal control microtissue. In addition, the stiffness of the microtissue can be measured through the ratio of the impedance (strain) of the microtissue over the pressure (stress). Liver fibrosis can be evaluated by measuring the stiffness of the tissue resulted from the excessive accumulation of a fibrous protein (e.g. collagen) induced by inflammation between liver cells. Impedance is proportional to the strain of the microtissue. So, precise regulation of the pressure can draw a stress-strain curve and the stiffness can be evaluated by observing the slope in that curve.

In the fatty liver and liver fibrosis evaluation system based on impedance above, the liver cell spheroid strain generating part contains the liver cell spheroid which is the impedance measurement target, and may be a space where the deformation of the liver cell spheroid may occur due to the external conditions. The liver cell spheroid strain generating part above comprises a first tube and a second tube installed in the inside of the first tube.

The first tube can have a hollow cylinder shape or a cylindrical shape. The second tube can also be in the shape of a hollow cylinder, or can be in the shape of a cylinder or the like.

The inner diameter of the first tube can be the same as the outer diameter of the second tube. The second tube can be installed in the inside of the first tube and at this time the outer surface of the second tube is contacted with the inner surface of the first tube. As the inner diameter of the first tube is the same as the outer diameter of the second tube, the second tube can be installed in the inside of the first tube. The second tube can be fixed in the center of the inside of the first tube. The first tube shares its side with the second tube longitudinally.

The inner diameter of the cylindrical second tube is preferably smaller than the diameter of the liver cell spheroid. Since the inner diameter of the cylindrical second tube is smaller than the diameter of the liver cell spheroid, the liver cell spheroid cannot pass through the second tube and thus can be fixed to one end of the second tube by decompression.

The liver cell spheroid strain generating part can comprise a third tube connected to one end of the first tube and controlling the position of the liver cell spheroid in the first tube by applying pressure in the first tube; and a fourth tube connected to another end of the first tube and controlling the position of the liver cell spheroid in the first tube by controlling pressure in the first tube.

The third tube above is in the shape of Y, which comprises a i tube connected to one end of the first tube; a ii tube connected to a syringe to adjust the position of the liver cell spheroid in the first tube by applying pressure in the first tube; and a iii tube containing an electrode material. The electrode material equipped in the iii tube is in the form of a coil-shaped wire, which can be extended into the inside of the i tube.

The fourth tube above is in the shape of Y, which comprises a I tube connected to another end of the first tube; a II tube connected to a barometer and a syringe pump; and a III tube containing an electrode material. The electrode material equipped in the III tube is in the form of a coil-shaped wire, which can be extended into the inside of the I tube.

As an example, the coil-shaped gold (Au) wire equipped in the iii tube can be extended into the inside of the i tube, and the coil-shaped platinum (Pt) wire equipped in the III tube can be extended into the inside of the I tube. When the gold and platinum wires are extended, the gold and platinum wires can share the buffer solution filled in the first tube, the second tube, the third tube and the fourth tube more easily, so that the changes in impedance can be measured accurately and reliably.

In the fatty liver and liver fibrosis evaluation system based on impedance above, the impedance measuring part comprises an impedance analyzer which is connected to the liver cell spheroid strain generating part above in order to measure the liver cell spheroid impedance. The impedance analyzer contains a working electrode and a counter electrode, and can be connected to the liver cell strain generating part. Particularly, the working electrode in the impedance analyzer can be connected to the electrode material included in the iii tube including the electrode material of the liver cell spheroid strain generating part, and preferably the electrode material can be connected to the coil-shaped gold wire protruding outwardly. The counter electrode in the impedance analyzer can be connected to the electrode material included in the III tube including the electrode material of the liver cell spheroid strain generating part, and preferably the electrode material can be connected to the coil-shaped platinum wire protruding outwardly.

The fatty liver and liver fibrosis evaluation system based on impedance above can evaluate fatty liver and liver fibrosis from the impedance measured at the impedance measuring part.

Particularly, the present invention provides, in an aspect of the present invention, a fatty liver and liver fibrosis evaluation system based on impedance which comprises a cylindrical first tube; a cylindrical second tube inserted in the first tube and having an outer surface in contact with the inner surface of the first tube and having a smaller length than that of the first tube; a Y-shaped third tube which is connected to one end of the first tube and comprises a i tube connected to one end of the first tube, a ii tube connected to a syringe to adjust the position of the liver cell spheroid in the first tube by applying pressure in the first tube, and a iii tube having a coil-shaped gold (Au) wire installed in the inside and one end of the gold wire is protruded to the outside; a Y-shaped fourth tube which is connected to another end of the first tube and comprises a I tube connected to another end of the first tube, a II tube connected to a barometer and a syringe pump through the tube, and a III tube having a coil-shaped platinum (Pt) wire installed in the inside and one end of the platinum wire is protruded to the outside; and an impedance analyzer including a working electrode connected to the gold wire protruded outwardly and a counter electrode connected to the platinum wire protruded outwardly.

In addition, the present invention provides a method for evaluating the test drug induced fatty liver comprising the following steps:

placing the liver cell spheroid treated with a test drug in the inside of the first tube of the fatty liver and liver fibrosis evaluation system based on impedance above;

locating the liver cell spheroid to contact with one end of the second tube and measuring the first impedance by using the impedance analyzer;

removing the liver cell spheroid from the second tube and measuring the second impedance by using the impedance analyzer;

obtaining the interior resistance, the exterior resistance and the capacitance of the liver cell spheroid based on the first impedance and the second impedance measured above; and

evaluating the test drug induced fatty liver by comparing the results above with those of the normal control.

In another aspect of the present invention, the present invention provides a method for evaluating the test drug induced fatty liver comprising the following steps:

placing the liver cell spheroid treated with a test drug in the inside of the first tube of the fatty liver and liver fibrosis evaluation system based on impedance above;

placing the liver cell spheroid in contact with one end of the second tube using the syringe connected to the ii tube;

fixing the liver cell spheroid in one end of the second tube using the syringe pump connected to the II tube and measuring the first impedance by using the impedance analyzer;

removing (detaching) the liver cell spheroid from the second tube using the syringe pump connected to the II tube and measuring the second impedance by using the impedance analyzer;

obtaining the interior resistance, the exterior resistance and the capacitance of the liver cell spheroid based on the first impedance and the second impedance measured above; and

evaluating the test drug induced fatty liver by comparing the results above with those of the normal control.

The method for evaluating the test drug induced fatty liver above can additionally include the step of placing the liver cell spheroid treated with a test drug in the inside of the first tube and arranging the first tube between the Y-shaped third tube and the Y-shaped fourth tube in order for the first tube to be connected to both of the third tube and the fourth tube.

The inner diameter of the second tube above is preferably smaller than the diameter of the liver cell spheroid, and the reason is as described above.

The method for evaluating the test drug induced fatty liver above is preferably executed as the liver cell spheroid strain generating part is filled with a buffer solution. For example, it is preferred that the method is performed as the first tube, the second tube, the third tube and the fourth tube are filled with a buffer solution.

The method for evaluating the test drug induced fatty liver above can also include the following steps:

judging the test drug to have a toxicity causing fatty liver when the interior resistance calculated from the liver cell spheroid treated with the test drug is bigger than that of the normal control;

judging the test drug to have a toxicity causing fatty liver when the exterior resistance calculated from the liver cell spheroid treated with the test drug is bigger than that of the normal control; and/or

judging the test drug to have a toxicity causing fatty liver when the capacitance calculated from the liver cell spheroid treated with the test drug is smaller than that of the normal control

In addition, the present invention provides a method for evaluating the test drug induced liver fibrosis comprising the following steps:

placing the liver cell spheroid treated with a test drug in the inside of the first tube of the fatty liver and liver fibrosis evaluation system based on impedance above;

locating the liver cell spheroid to contact with one end of the second tube and measuring the first impedance by using the impedance analyzer;

moving the liver cell spheroid into the inside of the second tube and measuring the second impedance by using the impedance analyzer;

moving the liver cell spheroid into the inside of the second tube and measuring the third impedance by using the impedance analyzer;

removing the liver cell spheroid from the second tube and measuring the fourth impedance by using the impedance analyzer;

obtaining the interior resistance, the exterior resistance and the capacitance of the liver cell spheroid based on the first impedance, the second impedance, the third impedance and the fourth impedance measured above; and

obtaining the stiffness of the spheroid from the pressure changes.

In another aspect of the present invention, the present invention provides a method for evaluating the test drug induced liver fibrosis comprising the following steps:

placing the liver cell spheroid treated with a test drug in the inside of the first tube of the fatty liver and liver fibrosis evaluation system based on impedance above;

placing the liver cell spheroid in contact with one end of the second tube using the syringe connected to the ii tube;

fixing the liver cell spheroid in one end of the second tube using the syringe pump connected to the II tube and measuring the first impedance by using the impedance analyzer;

moving the liver cell spheroid into the inside of the second tube using the syringe pump connected to the II tube and measuring the second impedance by using the impedance analyzer;

moving the liver cell spheroid into the inside of the second tube using the syringe pump connected to the II tube and measuring the third impedance by using the impedance analyzer;

removing (detaching) the liver cell spheroid from the second tube using the syringe pump connected to the II tube and measuring the fourth impedance by using the impedance analyzer;

obtaining the interior resistance, the exterior resistance and the capacitance of the liver cell spheroid based on the first impedance, the second impedance, the third impedance and the fourth impedance measured above; and

obtaining the stiffness of the spheroid from the pressure changes.

The method for evaluating the test drug induced liver fibrosis can additionally include the step of evaluating the test drug induced liver fibrosis by comparing the results with those of the control group.

In the method for evaluating the test drug induced liver fibrosis, the liver cell spheroid is fixed in one end of the second tube using the syringe pump connected to the II tube and then the first impedance is measured by using the impedance analyzer. Then, before removing the liver cell spheroid from the second tube using the syringe pump connected to the II tube, the degree of decompression is changed to measure the impedance additionally.

For example, the step of measuring the first impedance can additionally comprise the following sub steps:

measuring the 1-1 impedance by setting the pressure at −10 mPa using a syringe pump;

measuring the 1-2 impedance by setting the pressure at −15 mPa using a syringe pump; and

measuring the 1-3 impedance by setting the pressure at −20 mPa using a syringe pump.

As stronger the pressure is applied, the liver cell spheroid is fixed more tightly in one end of the second tube. As deeply the spheroid is entering in the inside of the second tube, the strain increases and accordingly the impedance increases. If the stiffness of the liver cell spheroid increases by a liver fibrosis inducing drug, the impedance rate over the stress would decrease.

The fatty liver and liver fibrosis evaluation system based on impedance provided in one aspect of the present invention uses 3-dimensional liver microtissues so that the test drug induced fatty liver and liver fibrosis can be analyzed with non-invasive and highly reproducible manner, which is supported by the following examples and experimental examples described hereinafter.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Preparative Example 1: Formation of Liver Microtissue (MT)

1. Completely differentiated HepaRG hepatocyte strain was obtained at the density of 4,000,000 cells/100 uL.

2. A sample was prepared according to the manual of C253000 provided by Microfit Co. 10 μL of cell suspension #1 was distributed in each well to allow 400,000 HepaRG cells to be placed per well. This step can be performed using a 3D cell preparation method such as a micropattern plate method or a hanging drop method.

3. The cells were cultured for 3 days and the cell culture medium was replaced every day.

4. On day 4, the microtissue (MT) was distributed in a 96-well plate (Ultra-Low attachment surface multiple well plate, Corning) using a 100 uL blue tip.

### Example 1: Construction of Fatty Liver and Liver Fibrosis Evaluation System Based on Impedance

1. A plastic tip (second tube) having an outer diameter of 1.8 mm, an inner diameter of 0.4 mm and a length of 5 mm was placed in the center of a glass tube (first tube) having an outer diameter of 2 mm, an inner diameter of 1.8 mm and a length of 8 cm.

2. Two Y-shaped tubes (third tube and fourth tube, respectively) were prepared. At least 99% gold (Au) wire with a diameter of 0.5 mm and a length of 5 cm was placed in the shape of coil in one side (iii tube) of one Y tube (third tube), and at least 99% platinum (Pt) wire with a diameter of 0.5 mm and a length of 5 cm was placed in the shape of coil in one side (III tube) of the other Y tube (fourth tube). At this time, the ends of the gold (Au) and platinum (Pt) wires were exposed outside the Y tube and the ends of the tubes (iii tube and III tube) were sealed to block air and water flow.

3. The other end (II tube) of the Y tube containing the platinum (Pt) wire was connected to the pressure sensor and the syringe pump via tube, and the other end (ii tube) of the Y tube containing the gold (Au) wire was connected to a 1 mL syringe in order to regulate the position of the liver microtissue (MT) manually.

4. Each Y tube was filled with PBS buffer.

### Experimental Example 1: Evaluation of Drug Induced Fatty Liver Toxicity

(following 1 to 4 of Example 1)

5a. The liver microtissue (MT) distributed in the Ultra-Low attachment surface multiple well plate was treated with 0.5 M oleic acid, a fatty liver toxicity inducing drug.

6a. A 200 μL pipette was inserted in the end of the first glass tube of Example 1 and the liver microtissue (MT) and the culture medium were sucked through the other end to fill the glass tube with preventing air bubble formation.

7a. The glass tube was inserted between the two Y tubes (third tube and fourth tube) of Example 1 and Example 4 with preventing air bubble formation.

8a. Air bubbles in the liquid were removed and let the wire of each Y tube shared the same liquid.

9a. The working electrode/sensing electrode of the potentiostat or impedance analyzer was connected to the gold (Au) wire protruding out of the Y tube (iii tube) and the reference/counter electrode was connected to the platinum (Pt) wire protruding out of the other Y tube (III tube).

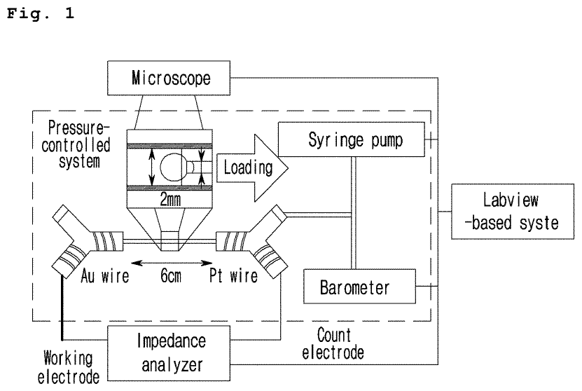

FIG. 1 presents a schematic diagram illustrating the image of the fatty liver and liver fibrosis evaluation system based on impedance provided in one aspect of the present invention.

10a. The liver microtissue (MT) was placed at the end of the plastic tip by using a 1 mL syringe.

11a. The pressure was set at −5 mPa by using the syringe pump and the liver microtissue (MT) was fixed at the plastic tip by the pressure difference.

12a. Alternating current impedance was measured at a fixed voltage of 50 mV from 10⁶ Hz to 10⁻² Hz to measure the impedance of the liver microtissue (MT) and the PBS buffer.

13a. The pressure of the syringe pump was set at 10 mPa to allow the liver microtissue (MT) to be removed from the plastic tip.

14a. Impedance was measured at a fixed voltage of 50 mV from 10⁶ Hz to 10⁻² Hz to measure the impedance of the PBS buffer (background).

15a. The interior resistance, the exterior resistance and the capacitance of 12a and the resistance and the capacitance of 14a were measured by using a Fitting program (e.g. Zview).

16a. The interior resistance, the exterior resistance and the capacitance of the liver microtissue (MT) were compared with those of the normal control group. At that time, the normal control group indicates the liver microtissue (MT) not treated with oleic acid which was regarded as a fatty liver toxicity inducing drug.

17a. When fat was formed in the liver tissue by oleic acid, electric current was intervened and thus the internal resistance was increased. When cell size was increased, the external resistance value reflecting the intercellular space was increased. The capacitance reflects the stability of the cell membrane. So, if a test drug has toxicity, the capacitance would be reduced.

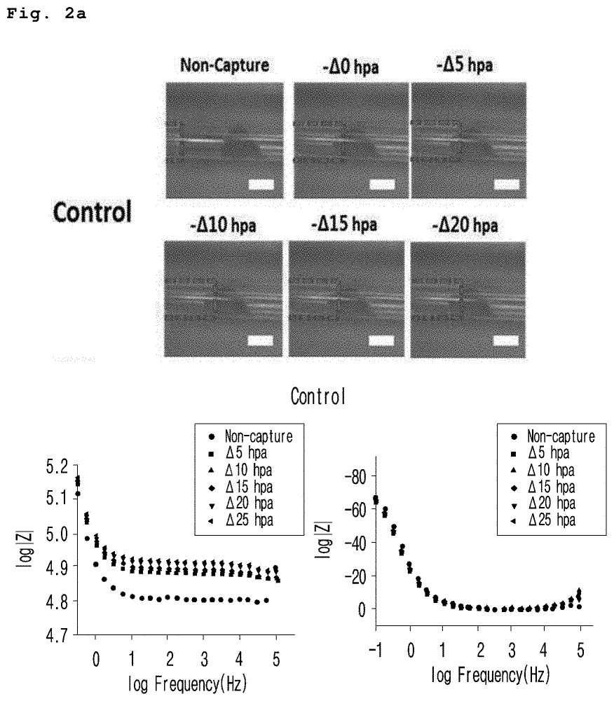

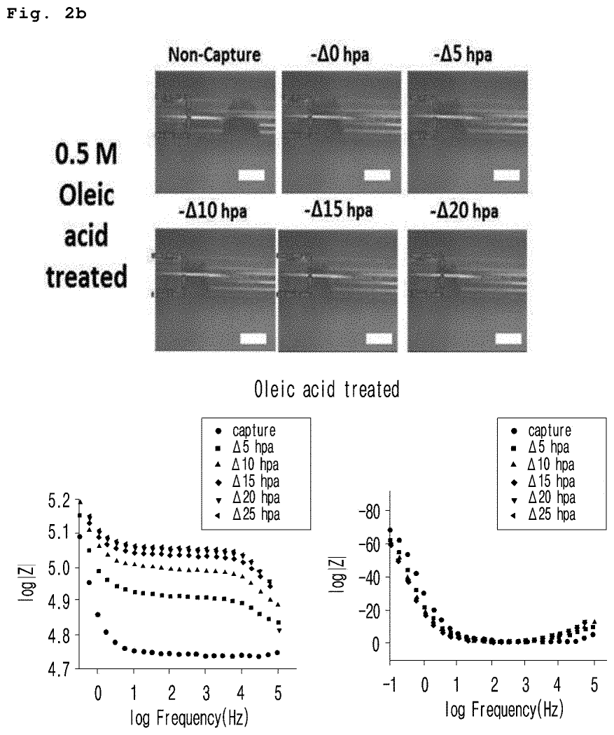

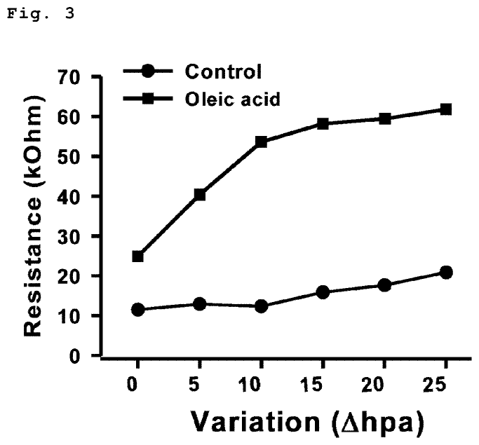

The results are shown in FIG. 2a, FIG. 2b and FIG. 3.

FIGS. 2a and 2b are a set of photographs and graphs showing the changes in deformation, impedance, and phase angle of the liver microtissue (MT) according to the pressure changes wherein FIG. 2a illustrates the control group and FIG. 2b illustrates the liver microtissue treated with oleic acid.

FIG. 3 is a graph illustrating the resistance pattern of the liver microtissue (MT) according to the pressure changes.

As shown in FIG. 2a, FIG. 2b and FIG. 3, fat was formed in the liver microtissue (MT) by oleic acid and therefore impedance and resistance were increased, compared with the normal control group.

From the results above, it was confirmed that the fatty liver and liver fibrosis evaluation system based on impedance provided in one aspect of the present invention uses 3-dimensional liver microtissues so that the test drug induced fatty liver and liver fibrosis can be analyzed with non-invasive and highly reproducible manner

### Experimental Example 2: Evaluation of Drug Induced Liver Fibrosis Toxicity

(following 1 to 4 of Example 1)

5b. The liver microtissue (MT) distributed in the Ultra-Low attachment surface multiple well plate was treated with TGF-beta, a liver fibrosis inducing drug.

6b˜12b. These steps were performed by the same manner as 6a˜12a of Experimental Example 1.

13b. The pressure of the syringe pump was set at −10 mPa, by which the liver microtissue (MT) was fixed more closely to the plastic tip.

14b. Alternating current impedance was measured at a fixed voltage of 50 mV from 10⁶ Hz to 10⁻² Hz to measure the impedance of the liver microtissue (MT) and the PBS buffer (background).

15b. The pressure of the syringe pump was set at −15 mPa, by which the liver microtissue (MT) was fixed more tightly to the plastic tip.

16b. Alternating current impedance was measured at a fixed voltage of 50 mV from 10⁶ Hz to 10⁻² Hz to measure the impedance of the liver microtissue (MT) and the PBS buffer (background). 17b. The pressure of the syringe pump was set at −20 mPa, by which the liver microtissue (MT) was fixed more tightly to the plastic tip.

18b. Alternating current impedance was measured at a fixed voltage of 50 mV from 10⁶ Hz to 10⁻² Hz to measure the impedance of the liver microtissue (MT) and the PBS buffer (background).

19b. The pressure of the syringe pump was set at 10 mPa to allow the liver microtissue (MT) to be removed from the plastic tip.

20b. Impedance was measured at a fixed voltage of 50 mV from 10⁶ Hz to 10⁻² Hz to measure the impedance of the PBS buffer (background).

21b. The interior resistances, the exterior resistances and the capacitances of 12b, 14b, 16b and 18b and the resistance and the capacitance of 20b were measured by using a Fitting program (e.g. Zview).

22b. The interior resistance, the exterior resistance and the capacitance measured above, except the PBS impedance, were compared with those of the control group. At that time, the normal control group indicates the liver microtissue (MT) not treated with TGF-beta which was regarded as a liver fibrosis inducing drug.

23b. Impedance increased in proportion to the strain of the liver microtissue (MT). So, the changes in strain according to the stress could be expressed as the slope of the pressure and impedance. When the stiffness of the liver microtissue (MT) was increased by such a liver fibrosis inducing drug, the ratio of strain over the stress was reduced.

We claim:

1. A fatty liver and liver fibrosis evaluation system based on impedance which comprises a liver cell spheroid strain generating part comprising a first tube; and a second tube inserted into the first tube and having a smaller length than that of the first tube; and an impedance measuring part comprising an impedance analyzer connected to the liver cell spheroid strain generating part to measure impedance of liver cell spheroid; and evaluating fatty liver and liver fibrosis through the impedance measured in the impedance measuring part.

2. The fatty liver and liver fibrosis evaluation system based on impedance according to claim 1, wherein the inner diameter of the first tube is the same as the outer diameter of the second tube and the inner diameter of the second tube is smaller than the diameter of the liver cell spheroid.

3. The fatty liver and liver fibrosis evaluation system based on impedance according to claim 1, wherein the liver cell spheroid strain generating part comprises a third tube connected to one end of the first tube and controlling the position of the liver cell spheroid in the first tube by applying pressure in the first tube; and a fourth tube connected to another end of the first tube and controlling the position of the liver cell spheroid in the first tube by controlling pressure in the first tube.

4. The fatty liver and liver fibrosis evaluation system based on impedance according to claim 3, wherein the third tube is in the shape of Y and comprises a i tube connected to one end of the first tube, a ii tube connected to a syringe to adjust the position of the liver cell spheroid in the first tube by applying pressure in the first tube, and a iii tube containing an electrode material.

5. The fatty liver and liver fibrosis evaluation system based on impedance according to claim 3, wherein the fourth tube is in the shape of Y and comprises a I tube connected to another end of the first tube, a II tube connected to a barometer and a syringe pump, and a III tube containing an electrode material.

6. The fatty liver and liver fibrosis evaluation system based on impedance according to claim 4, wherein the electrode material contained in the iii tube is in the form of a coil-shaped wire, and is extended into the inside of the i tube.

7. The fatty liver and liver fibrosis evaluation system based on impedance according to claim 5, wherein the electrode material contained in the III tube is in the form of a coil-shaped wire, and is extended into the inside of the I tube.

8. A method for evaluating the test drug induced fatty liver comprising:
placing the liver cell spheroid treated with a test drug in the inside of the first tube of the fatty liver and liver fibrosis evaluation system based on impedance of claim 1;
locating the liver cell spheroid to contact with one end of the second tube and measuring the first impedance by using the impedance analyzer;
removing the liver cell spheroid from the second tube and measuring the second impedance by using the impedance analyzer;
obtaining the interior resistance, the exterior resistance and the capacitance of the liver cell spheroid based on the first impedance and the second impedance measured above; and
evaluating the test drug induced fatty liver by comparing the results above with those of the normal control group.

9. The method for evaluating the test drug induced fatty liver according to claim 8, wherein the method is performed as the liver cell spheroid strain generating part is filled with buffer.

10. The method for evaluating the test drug induced fatty liver according to claim 8, wherein the method additionally includes judging the test drug to have a toxicity causing fatty liver when the interior resistance calculated from the liver cell spheroid treated with the test drug is bigger than that of the normal control group.

11. The method for evaluating the test drug induced fatty liver according to claim 8, wherein the method additionally includes judging the test drug to have a toxicity causing fatty liver when the exterior resistance calculated from the liver cell spheroid treated with the test drug is bigger than that of the normal control group.

12. The method for evaluating the test drug induced fatty liver according to claim 8, wherein the method additionally includes judging the test drug to have a toxicity causing fatty liver when the capacitance calculated from the liver cell spheroid treated with the test drug is smaller than that of the normal control group.

13. A method for evaluating the test drug induced liver fibrosis comprising:
placing the liver cell spheroid treated with a test drug in the inside of the first tube of the fatty liver and liver fibrosis evaluation system based on impedance of claim 1;
locating the liver cell spheroid to contact with one end of the second tube and measuring the first impedance by using the impedance analyzer;
pushing the liver cell spheroid in the inside of the second tube and measuring the second impedance by using the impedance analyzer;
pushing the liver cell spheroid in the inside of the second tube and measuring the third impedance by using the impedance analyzer;
removing the liver cell spheroid from the second tube and measuring the fourth impedance by using the impedance analyzer;
obtaining the interior resistance, the exterior resistance and the capacitance of the liver cell spheroid based on the first impedance, the second impedance, the third impedance and the fourth impedance measured above; and
obtaining the stiffness of the spheroid from the pressure changes.

14. The method for evaluating the test drug induced liver fibrosis according to claim 13, wherein the inner diameter of the second tube is smaller than the diameter of the liver cell spheroid.

15. The method for evaluating the test drug induced liver fibrosis according to claim 13, wherein the method is performed as the liver cell spheroid strain generating part is filled with buffer.
